SEQUENCE LISTING (2 sequences)

SEQ ID NO: 1 - RNA, 13 nt, Artificial Sequence
  Shine-Dalgarno sequence.
gaucaccuccuua

SEQ ID NO: 2 - DNA, 13 bp, Artificial Sequence
  DNA complement of Shine-Dalgarno sequence.
taaggaggtgatc

(19) United States
(12) Patent Application Publication
(10) Pub. No.: US 2003/0073078 A1
(43) Pub. Date: Apr. 17, 2003

(54) Method for the recognition of translating genetic coding sequences

(76) Inventors: David Ian Rosnick, Raleigh, NC (US); Donald L. Bitzer, Cary, NC (US); Mladen A. Vouk, Cary, NC (US); Eleboba Eni May, Raleigh, NC (US)

Correspondence Address: MYERS BIGEL SIBLEY & SAJOVEC, PO BOX 37428, RALEIGH, NC, 27627, US

(21) Appl. No.: 09/905,289

(22) Filed: Jul. 13, 2001

Related U.S. Application Data
(60) Provisional application No. 60/219,887, filed on Jul. 21, 2000.

(51) Int. Cl. C12Q 1/68; G06F 19/00; G01N 33/48; G01N 33/50

(52) U.S. Cl. 435/6; 702/20

(57) ABSTRACT

Computer-based methods are useful for screening a nucleic acid sequence for efficient translation in a predetermined host. These methods involve providing a 3′-end terminal ribosomal nucleic acid (rRNA) sequence, providing a substrate nucleic acid sequence, and detecting the presence or absence of a three-base binding strength (i.e., binding energy) periodic cycle and/or phase between the ribosomal nucleic acid sequence and the substrate nucleic acid sequence through the substrate nucleic acid sequence. The presence of the three base periodic binding strength cycle and/or correct phase through the substrate nucleic acid sequence indicates that the substrate nucleic acid sequence is a candidate for efficient translation in the host.

### RELATED APPLICATIONS

This application claims the benefit of provisional application serial No. 60/219,887, filed Jul. 21, 2000, the disclosure of which is incorporated by reference herein in its entirety.

### FIELD OF THE INVENTION

The present invention concerns computer-based methods for determining whether a given nucleic acid segment is a coding segment, or for determining whether such a nucleic acid segment will be efficiently translated in an mRNA by a host organism such as a transgenic host organism.

### BACKGROUND OF THE INVENTION

Considerable effort has been devoted to mapping the genome of numerous organisms. However, the mapping of a genome does not identify all potential coding regions within that genome (i.e., regions that code for a protein or peptide of interest). Accordingly, it is useful to provide techniques for screening genetic sequence data to determine whether the sequences are coding sequences.

A related problem is presented when one wishes to produce a particular protein or peptide in a transgenic organism. In such a case, while the nucleic acid of interest may encode the protein or peptide of interest in its native species, the nucleic acid may not be efficiently expressed in the transgenic host organism. Currently, the optimization of nucleic acids for transgenic expression is a largely empirical process, requiring much laboratory experimentation. It would be extremely useful to provide predictive techniques that help indicate whether a particular nucleic acid will at least be efficiently translated as mRNA in a transgenic host.

Analyses of nucleic acid features such as coding length and presence of particular sequences upstream from a start codon are often employed to identify true coding sequences from a theoretical standpoint. One example is the Shine-Dalgarno (SD) sequence. It is an identifier near the 3′ end of the 16S rRNA ribosomal subunit which frequently displays a strong homology between its Watson-Crick complement and regions upstream from the coding sequences (J. Shine and L. Dalgarno, Proc. Natl. Acad. Sci. 71, 1342-1346 (1974)). Research suggests that the distribution of bases within a sequence relates to translocation and frame shifting (R. B. Weis et al., EMBO J. 7, 1503-1507 (1988)). Though extensively studied and modestly employed in coding detection, the idea of phase bias as a necessary condition of proper translation remains controversial (See, e.g., J. F. Curran and B. L. Gross, J. Mol. Biol. 235, 389-395 (1994)).

Accordingly, there remains a need for new ways to identify coding sequences, and there remains a need for new ways to determine whether a given coding sequence will be adequately or efficiently translated in a host organism.

### SUMMARY OF THE INVENTION

A first aspect of the present invention is a computer-based method for screening a nucleic acid sequence for efficient translation in a predetermined host (e.g., a heterologous or homologous host). The method comprises the steps of: (i) providing a 3′-end terminal ribosomal nucleic acid (rRNA) sequence (The ribosomal nucleic acid sequence preferably comprising a continuous segment 3′ to the first hairpin of the rRNA (e.g., all or part of the exposed tail region), and is preferably of from 5 or 6 to 15 or 20 bases of the first 15 or 20 bases closest to the 3′ end of the 16S or 18S rRNA ribosomal subunit of the host); (ii) providing a substrate nucleic acid sequence; and (iii) detecting the presence or absence of a three-base binding strength (i.e., binding energy) periodic cycle and/or phase between said ribosomal nucleic acid sequence and said substrate nucleic acid sequence through said substrate nucleic acid sequence, the presence of said three base periodic binding strength cycle and/or correct phase through said substrate nucleic acid sequence indicating said substrate nucleic acid sequence is a candidate for efficient translation in said host. More particularly, the method comprises the steps of:

(a) providing a 3′-end terminal ribosomal nucleic acid (rRNA) sequence as described above;

(b) providing a substrate nucleic acid sequence;

(c) determining a first binding strength (e.g., binding energy) of said ribosomal nucleic acid sequence to said substrate nucleic acid sequence at a first alignment (or position) of interest in the substrate;

(d) determining a second binding strength of said ribosomal nucleic acid sequence to said substrate nucleic acid sequence at a second alignment of interest; wherein said second substrate alignment of interest is one base downstream from said first alignment of interest;

(e) determining a third binding strength of said ribosomal nucleic acid sequence to said substrate nucleic acid sequence at a third alignment of interest, wherein said third substrate alignment of interest is two bases downstream from said first alignment of interest;

(f) successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;

(g) generating a binding strength pattern from said first through third binding strengths determined in said successively repeated steps (c) through (e); and

(h) detecting the presence or absence of a three-base binding strength periodic cycle and/or phase through said substrate nucleic acid sequence from said binding strength pattern, the presence of said three base periodic binding strength cycle and/or correct phase through said substrate nucleic acid sequence indicating said substrate nucleic acid sequence is a candidate for efficient translation in said host.

By “downstream” is meant either direction herein, including the natural direction of translation (e.g., from the 3′ towards 5′ end of the ribosomal nucleic acid; or from the 5′ toward the 3′ end of the substrate nucleic acid), or the opposite direction of translation. The natural direction of translation is currently preferred. Also note that by “successively repeating” herein is meant that any order (e.g., forward, backward, sequentially, random positions, random blocks) may be employed as long as all necessary data is stored and appropriately identified for subsequent processing and analysis.

In the case where it is desired to optimize the translation of a heterologous or transgenic nucleic acid in a host (including a native host), the method may further comprise the steps of:

(j) determining the sufficiency of said translation efficiency from said quantitative indicator; and then, in the absence of sufficient translation efficiency;

(k) replacing at least one base in said substrate nucleic acid sequence with a different base (e.g., based upon a table of alternate codons coding for the same amino acid) base to produce a subsequent substrate nucleic acid sequence different from said previous substrate nucleic acid sequence, and encoding the same protein or peptide; and

(l) repeating steps (c) through (j) above with said subsequent substrate nucleic acid sequence (e.g., repeating steps (c) through (k) until a nucleic acid sequence having sufficient efficiency of translation in said host is identified).

A second aspect of the present invention is a computer-based method for screening a nucleic acid sequence for the presence of at least one coding sequence therein. The method comprises the steps of: providing a substrate nucleic acid sequence from a predetermined host; providing a ribosomal nucleic acid sequence as described above for said host; and then detecting the presence or absence of a three-base binding strength periodic cycle and/or phase between said substrate nucleic acid sequence and said ribosomal nucleic acid sequence in at least one portion of the said substrate nucleic acid sequence, the presence of said three base periodic binding strength cycle and/or correct phase through said substrate nucleic acid sequence indicating said at least one portion of said nucleic acid sequence is a coding portion in said host. More particularly, the method comprises the steps of:

(a) providing a substrate nucleic acid sequence from a predetermined host;

(b) providing a ribosomal nucleic acid sequence as described above for said host;

(c) determining a first binding strength of said ribosomal nucleic acid sequence to said substrate nucleic acid sequence at a first alignment of interest;

(d) determining a second binding strength of said ribosomal nucleic acid sequence to said substrate nucleic acid sequence at a second alignment of interest; wherein said second alignment of interest is one base downstream from said first alignment of interest;

(e) determining a third binding strength of said ribosomal nucleic acid sequence to said substrate nucleic acid sequence at a third alignment of interest, wherein said third alignment of interest is two bases downstream from said first alignment of interest;

(f) successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;

(g) generating a binding strength pattern from said first through third binding strengths determined in said successively repeated steps (c) through (e); and

(h) detecting the presence or absence of a three-base binding strength periodic cycle and/or phase in at least one portion of the said substrate nucleic acid sequence from said binding strength pattern, the presence of said three base periodic binding strength cycle and/or correct phase through said substrate nucleic acid sequence indicating said at least one portion of said nucleic acid sequence is a coding portion in said host.

The method may optionally further include the step of:

(i) determining a start region or location and a stop region or location for said coding portion within said substrate nucleic acid sequence.

In the foregoing methods, the detecting step may further comprise the step of determining the strength of said periodic signal. Further, the methods may further comprise generating a quantitative indicator of translation efficiency from the strength of said periodic signal. Such a quantitative indicator may be numerical, graphic, text or the like. Note that quantitative values of phase and binding strength are typically computed using different pattern generation and presentation methods.

In certain preferred embodiments, the foregoing methods further comprise the steps of detecting a phase shift in said three base periodic cycle; and determining the presence of a frame shift in said substrate nucleic acid sequence from said phase shift, so that said substrate nucleic acid sequence remains a candidate for efficient translation in said host in the presence of said phase shift. Note that a phase shift (if permanent) implies a frame shift which may translate efficiently. A constantly changing phase implies possible and true frame shifts which may destabilize the translation, stop it, or make it less efficient. Note the frame shift may occur when there is a sudden change in phase, or when a constantly changing phase shift exceeds a certain threshold change rate (but it may be stable below that threshold despite that it changes continuously).

Further aspects of the present invention include computer systems for carrying out the aforeasaid methods, along with computer program products comprised of a computer usable storage medium having computer readable program code embodied in the medium for programming or implementing such systems.

The present invention is explained in greater detail in the drawings herein and the specification set forth below.

### BRIEF DESCRIPTION OF THE DRAWINGS

FIG. 1 is a flow chart showing the general analysis procedure for the current invention.

FIG. 2 is a flow chart of the Sequence Optimization step of FIG. 1.

FIG. 3 is a flow chart of the Fourier Analysis calculation of FIG. 1.

FIG. 4 is a flow chart of the Energy History calculation of FIG. 1.

FIG. 5 is a flow chart of the Phase Analysis calculation of FIG. 1.

FIG. 6 is a flow chart of the Binding Energies calculation of FIGS. 3 and 4.

FIG. 7. Average binding strength—near start.

FIG. 8. Average binding strength—downstream.

FIG. 9. Power spectral density—coding regions.

FIG. 10A. Example power spectrum for single coding sample.

FIG. 10B. Example power spectrum for a single non-coding sample.

FIG. 11. aceF energy differential by alignment.

FIG. 12. prfb energy differential by alignment.

FIG. 13. Vector energy output for pCZ105.

FIG. 14. Vector energy output for pCZ110.

FIG. 15. Example memory register contents.

FIG. 16. Example energy differential by position.

FIG. 17A. Example energy for perfect memory.

FIG. 17B. Example energy differential by position for perfect memory.

### DETAILED DESCRIPTION OF THE PREFERRED EMBODIMENTS

The present invention now will be described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As will be appreciated by one of skill in the art, the present invention may be embodied as a method, data processing system, or computer program product. Accordingly, the present invention may take the form of an entirely hardware embodiment, an entirely software embodiment, or an embodiment combining software and hardware aspects. Furthermore, the present invention may take the form of a computer program product on a computer-usable storage medium having computer readable program code means embodied in the medium. Any suitable computer readable medium may be utilized including, but not limited to, hard disks, CD-ROMs, optical storage devices, and magnetic storage devices.

The present invention is described below with reference to flowchart illustrations of methods, apparatus (systems), and computer program products according to an embodiment of the invention. It will be understood that each block of the flowchart illustrations, and combinations of blocks in the flowchart illustrations, can be implemented by computer program instructions (or code means). These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function specified in the flowchart block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

The substrate nucleic acid may be, as noted above, from the same species as the host (homologous to said host), or may be from a different species as the host (heterologous to the host). In the latter case, the substrate nucleic acid is transgenic with respect to the host and is from a different source than the ribosomal nucleic acid sequence. The substrate nucleic acid sequence may be natural or synthetic. For example, the substrate nucleic acid sequence may encode a predetermined protein or peptide, such as human insulin, bovine growth hormone, human growth hormone, human erythropoietin. The substrate nucleic acid and its encoded protein or peptide may thus be human, mammalian (cow, sheep, pig, horse, etc.), or other animal such as bird, plant (e.g., vascular plant), bacterial, or of any other suitable species of origin. The substrate nucleic acid may be RNA (mRNA) or DNA, with transcription employed as necessary and (when introns are present) splicing steps employed as necessary to carry out the method. The substrate nucleic acid sequence may be of any suitable length (e.g., 20, 40, 60 or 80 nucleic acids to 1,000, 10,000 or 20,000 or more), and may exist either as a discrete segment or may exist within a longer sequence that is undergoing analysis.

The present invention can be implemented with both prokaryotic and eukaryotic hosts or host organisms. Numerous prokaryotic and eukaryotic ribosomal RNA sequences are known which can be used to carry out the present invention. For example, http://www.ncbi.nlm.nih.gov/ will link the user to the national database where the user can type in the key words “Eukaryota ribosomal RNA” or “Eukaryota rRNA” and get the sequence for sequenced eukaryotic rRNAs. There are currently over 50,000 partial and full sequences in the database.

As explained in greater detail below, one embodiment of the present invention includes predicting the potential for proper translation of said substrate nucleic acid sequence in said host using the said already determined binding strengths of said ribosomal nucleic acid sequence to said substrate nucleic acid sequence.

A variety of features may be incorporated into the present invention and the various steps performed in a variety of different ways, as will be apparent to those skilled in the art. For example, the step (g) of generating a binding strength pattern as noted above may comprise one or more calculating step(s), including (i) calculating a summation of all said binding strengths, (ii) calculating a summation of said first, second, or third binding strengths, (iii) calculating an integral of all said binding strengths or of said first, second, or third binding strengths, (iv) calculating a partial integral of all said binding strengths or of said first, second, or third binding strengths, (v) calculating a running average of all said binding strengths or of said first, second, or third binding strengths, and (vi) calculating transforms (e.g., Fourier transforms or transforms by other methods such as coding-based or wavelet methods) of all said binding strengths or of said first, second, or third binding strengths.

Note that the term “running average” as used herein is intended to be construed generally and to include running averages, moving averages, weighted moving averages and weighted running averages. Weighted moving averages are currently preferred.

The step (h) of detecting the presence or absence of a three-base binding strength periodic cycle as noted above may also comprises one or more calculating step(s), including (i) calculating a summation of all said binding strengths, (ii) calculating a summation of said first, second, or third binding strengths, (iii) calculating an integral of all said binding strengths or of said first, second, or third binding strengths, (iv) calculating a partial integral of all said binding strengths or of said first, second, or third binding strengths, (v) calculating a running average of all said binding strengths or of said first, second, or third binding strengths, and (vi) calculating transforms (e.g., Fourier transforms or transforms by other methods such as coding-based or wavelet methods) of all said binding strengths or of said first, second, or third binding strengths.

The step (f) of successively repeating steps (c) through (e) may further comprise the step of: calculating a cumulative energy for each said first, second, or third alignment of interest.

The methods may include the step of calculating a cumulative energy differential for a portion of said substrate nucleic acid sequence.

The methods may include the step of calculating a power spectrum magnitude of said binding strengths of said ribosomal nucleic acid sequence to said substrate nucleic acid sequence for a portion of said substrate nucleic acid sequence.

The methods may include the step of calculating a mean binding strength of said ribosomal nucleic acid sequence to said substrate nucleic acid sequence for a portion of said substrate nucleic acid sequence.

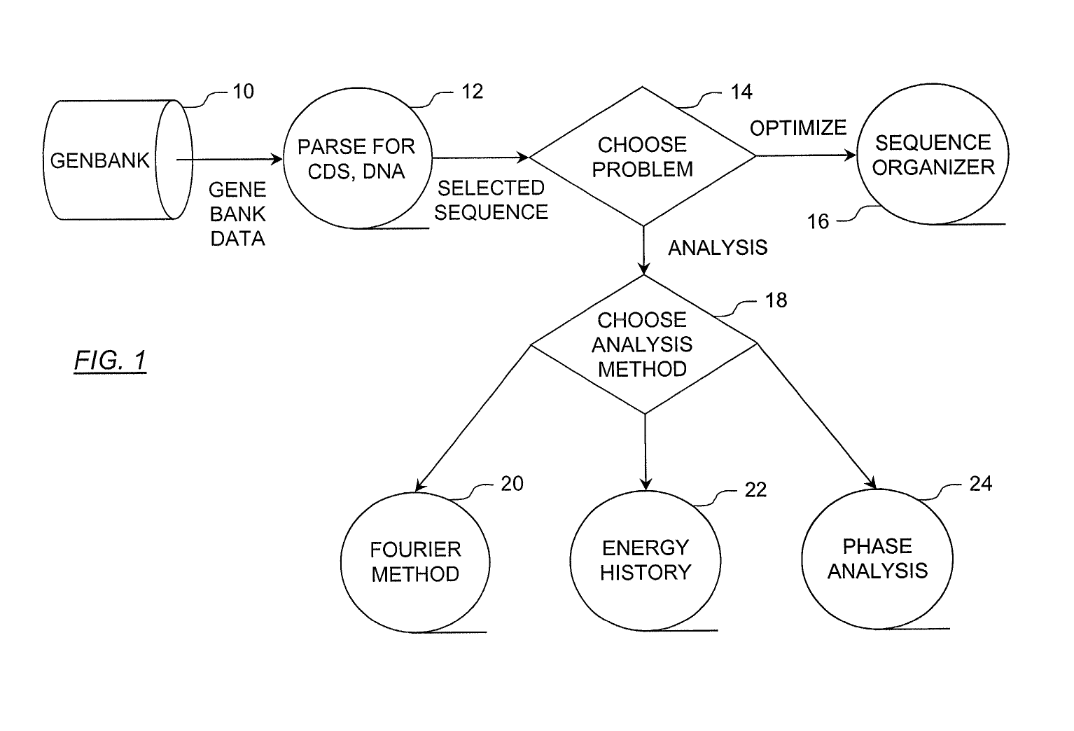

FIGS. 1-6 illustrate the method of the present invention in flow chart form. In a preferred embodiment, the method is implemented as C routines and MATLAB analysis tools on a Sun Sparc Ultra-5 workstation running Solaris 2.5.1. Note that hardware other than a Sun workstation and mathematical software other than MATLAB may also be used.

FIG. 1 is a flow chart showing the general analysis procedure for the current invention. Data procured from an available source, such as a genetic bank 10, is parsed for CDS and DNA information 12. After a sequence of genetic material has been selected 14, the researcher elects whether to optimize the sequence 16 or to analyze it further 18. If the researcher chooses to analyze the sequence, three different methods of analysis are available to obtain different data about the sequence. The three methods are Fourier Analysis 20 (or other transform analysis such as coding-based or wavelet methods), Energy History Analysis 22, and Phase Analysis 24.

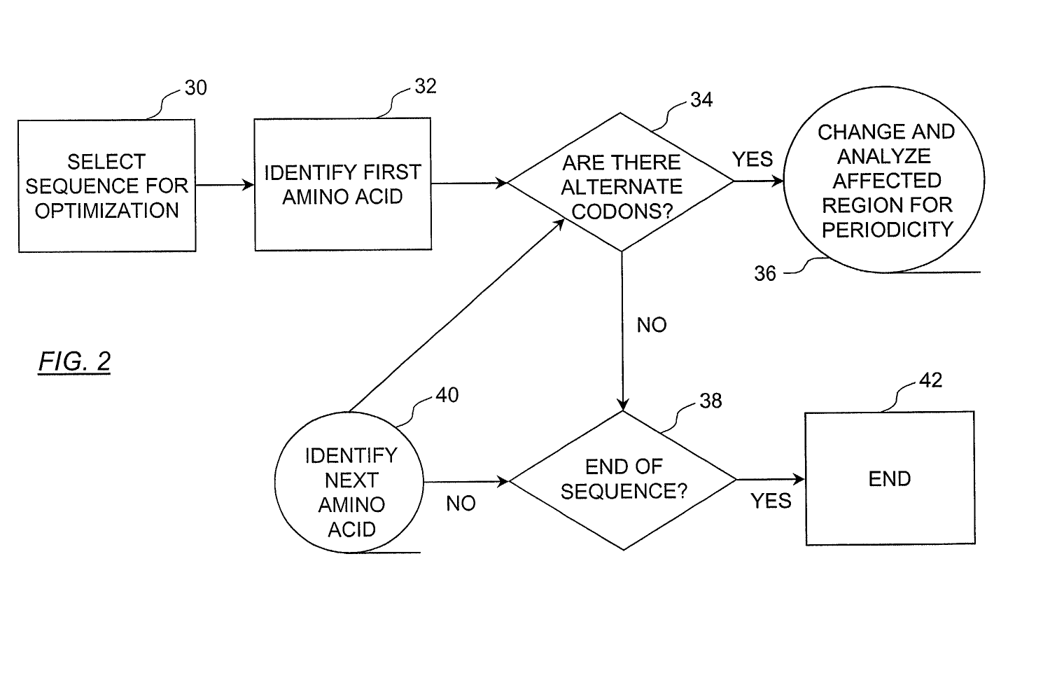

FIG. 2 is a flow chart of the Sequence Optimization step 16 of FIG. 1. Once a sequence has been selected for optimization 30, the first amino acid must be identified 32. If there are alternate codons 34, the affected region is analyzed for periodicity 36. Once there are no alternate codons, it is determined whether the end of the sequence has been reached 38. If the end has not been reached, the next amino acid is identified 40 and the process begins again. Once the end is reached, the optimization process is at an end 42.

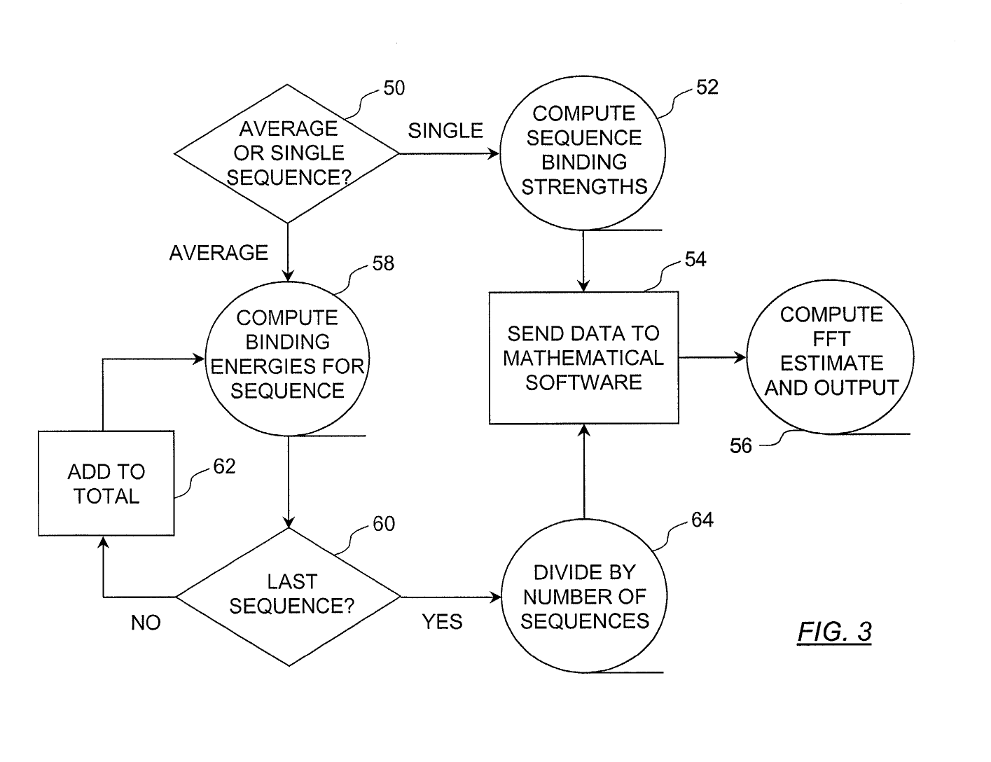

FIG. 3 is a flow chart of the Fourier Analysis calculation 20 of FIG. 1. The first choice 50 is whether a single sequence or an average of multiple sequences is being analyzed. If a single sequence, the binding energies for the sequence are computed 52, and then that data is fed into MATLAB computer routines 54, which are used to compute and output an FFT estimate 56. If an average of multiple sequences is being analyzed, the binding energies for one sequence are computed 58. The binding energies for each successive sequence are also computed 60 and added to the total 62 until binding energies have been computed for all the sequences. Then the total is divided by the number of sequences 64, and the result is fed into MATLAB computer routines 54, which are used to compute and output an FFT estimate 56.

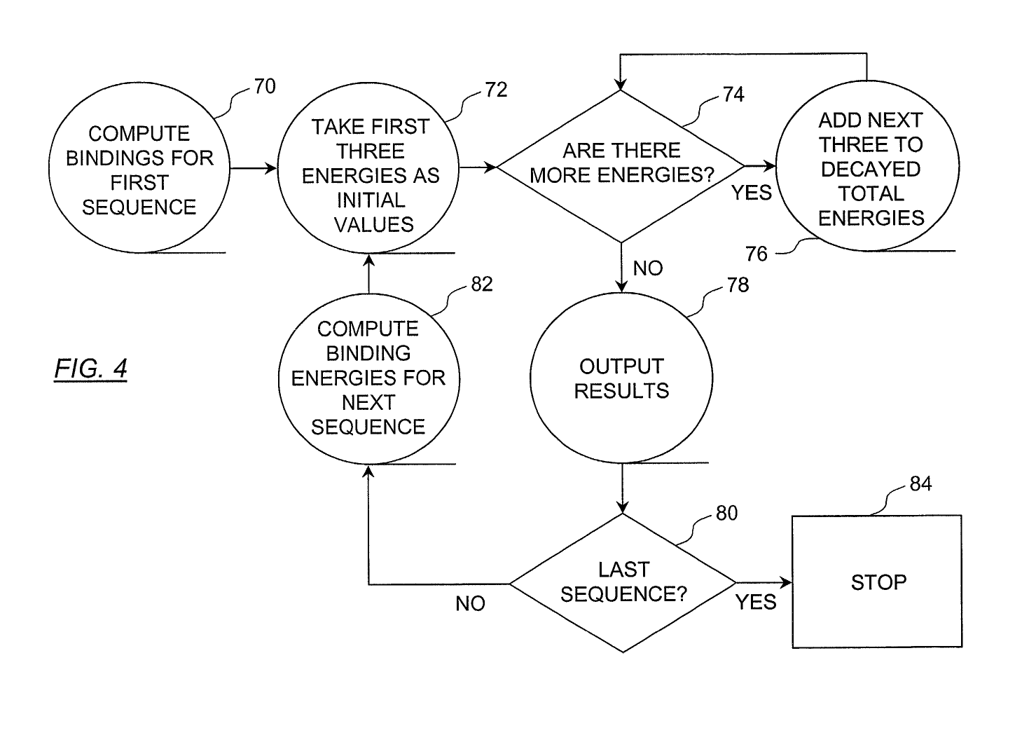

FIG. 4 is a flow chart of the Energy History calculation 22 of FIG. 1. First, the binding energies of the first sequence are computed 70. The first three energies are taken as initial values 72. If there are more energies 74, the next three are added to a decayed total energy. The results are made available once there are no more energies 78. If there are more sequences 80, the binding energies for the next sequence are computed 82, the first three energies are taken as initial values 72, and the process is repeated. Once there are no more sequences, the energy history calculation is complete 84.

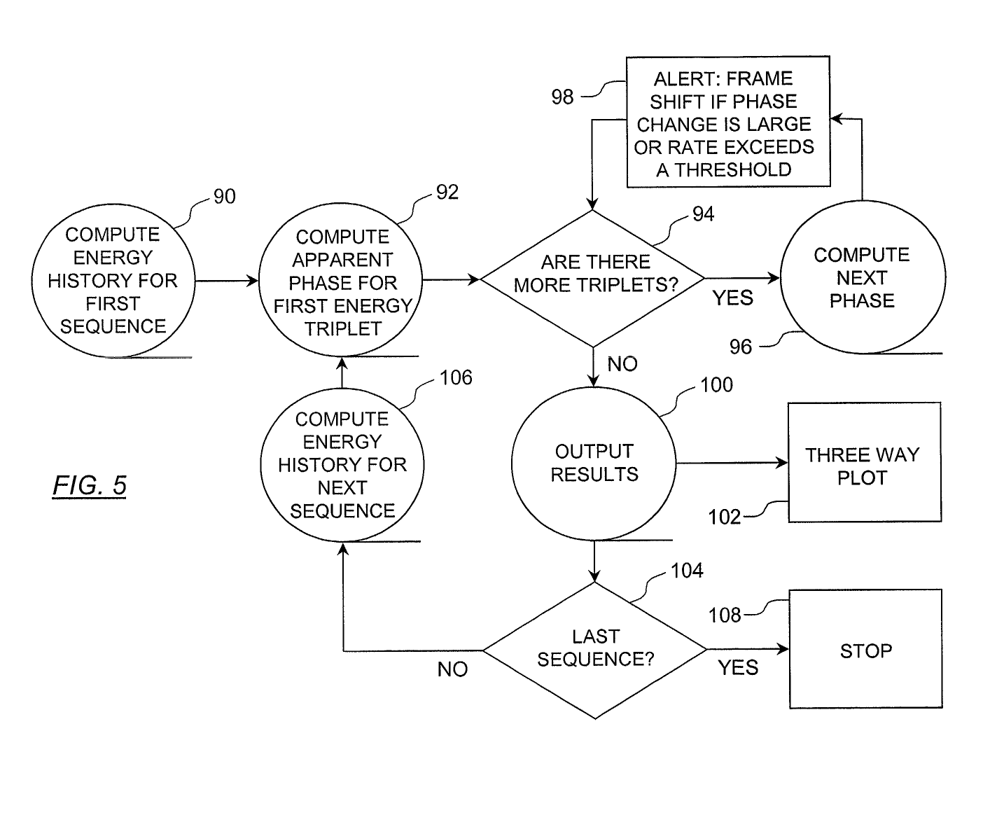

FIG. 5 is a flow chart of the Phase Analysis calculation 24 of FIG. 1. First, the energy history for the first sequence is computed 90. Then the apparent phase of the first energy triplet is computed 92. If there are more triplets 94, each successive next phase is computed 96 until there are no more triplets. If at some point within this iteration the phase change is large or constant rate of change of phase exceeds a threshold, this serves as an alert that there is a frame shift 98. Once there are no more triplets, the results are made available 100. A three-way plot can be made at this point 102. If the sequence for which the phase analysis was calculated is not the last sequence 104, the energy history for the next sequence is computed 106, and the entire process is repeated until there are no more sequences 108.

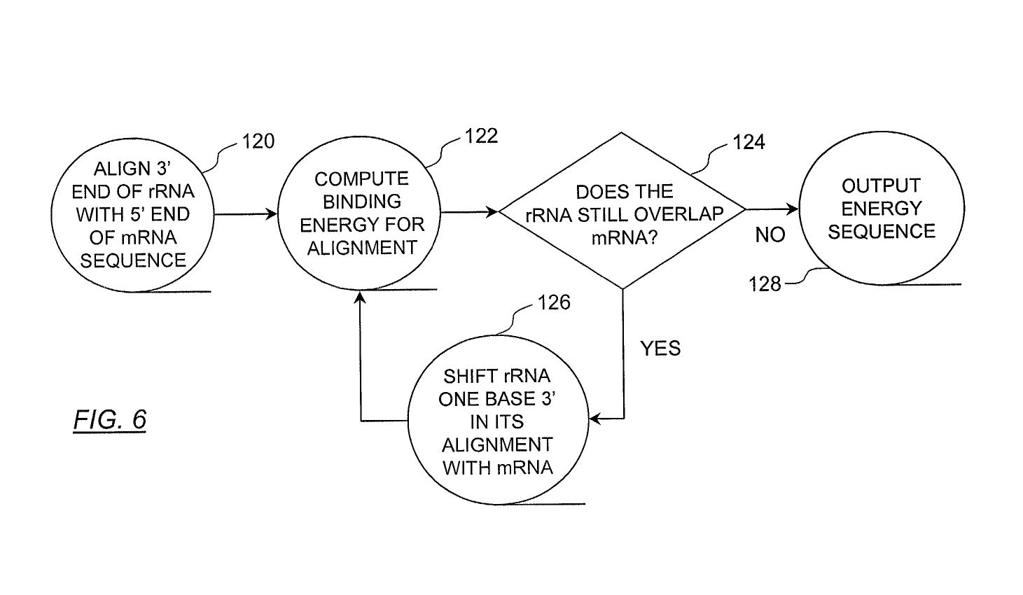

FIG. 6 is a flow chart of the Binding Energies calculation of FIGS. 3 and 4. The 3′ end of rRNA is aligned with the 5′ end of the mRNA sequence 120. The binding energies for the alignment are computed 122, and if the rRNA still overlaps the mRNA 124, the rRNA is shifted one base 3′ in its alignment with the mRNA 126 and the binding energies for the new alignment are calculated 122. This process is repeated until the rRNA no linger overlaps the mRNA, at which point the energy sequence results are made available 128.

As one example of the instant invention, the Shine-Dalgarno' (SD) sequence we consider here the sequence GAUCACCUCCUUA (SEQ ID NO:1), read 5′-3′ from the 16S, and its DNA complement (cSD) TAAGGAGGTGATC (SEQ ID NO:2). Homology between cSD and upstream mRNA and free energy release due to Watson-Crick binding between SD and the upstream sequences have both been shown to relate to translation rates of the coding regions to which they correspond. The presence of the cSD in these region is not, however, the sole translation indicator (P. Bermel, Eni, M. Vouk and D. Bitzer, On the import of the Shine-Dalgarno series to the expression of mRNA sequences, Department of Computer Science, North Carolina State University).

If upstream presence of the sequence is necessary for initiation of translation, then elongation may mandate continued affinity between the 16S and mRNA well into a coding sequence. Experimental research into downstream homology indicates that the Shine-Dalgarno sequence impacts the efficiency of a shifty stop (See, e.g., R. B. Weiss et al., EMBO J. 7, 1503-1507 (1988)). Here, those affinities are examined through free energy calculations. Previous examinations have been generally limited to the upstream regions (T. Schurr et al., Nucleic Acids. Res. 21, 4019-4023 (1993)), or simpler base preferences (E. Trifonov, J. Mol. Biol., 194, 643-652 (1987); E. Trifonov, Biochimie, 74, 357-362 (1992)). We consider the entire coding region, and eventually, the entire Escherichia coli genome. Free energy calculations are performed based on calculations used in the formation of mRNA secondary structure (B. Lewin, Genes VI, Oxford University Press, New York, N.Y. 1997). For every coding sequence, the minimum free energy at every base position is computed. The average energy at each position on the messenger strand taken over 2095 coding sequences produces an obvious three base periodic signal down the coding region. Preference for one phase of the signal relative to the start codon is also examined. The RF-2 gene is singled out as particularly demonstrative of the phase necessity, as the energy pattern shifts along with the natural+1 frameshift.

Although any unequal distribution of the bases among the three positions should result in a correlation of the free energy values, the observed base preference broadly corresponds to the distribution expected for three-periodic homology with the SD sequence. Because of the high noise level in the energy pattern, it is suggested that a memory mechanism exists within the ribosome to monitor the pattern over the length of the strand.

It is then suggested that the SD portion of the 16S rRNA plays a role as the ribosome maintains frame through the coding region, and therefore on downstream base preference.

Methods. Data we used in these computer experiments is taken from the National Institute of Health genome database, GenBank (D. Benson et al. GenBank, Nucleic Acids Res., 26, 1-7 (1988)).

We developed C computer program to read in GenBank datafiles containing a listing of a DNA sequence, annotated with actual and proposed coding sequences, and to then compute the binding energies between the sequence and an input mask from that data. The data is then analyzed and results visualized using simple Matlab routines.

We presuppose that the E. coli mRNA transcribes exactly from the DNA sequence listed in the GenBank datafile accession code U00096. We also assume the accuracy and completeness of the GenBank data. This is especially important with regard to the annotation of coding sequences. Particularly, for the purposes of this research, a coding sequence is any region annotated “CDS”′, although distinctions are made for uncertain sequences listed as “hypothetical,” “putative,” or simply unknown ORF.

We employ a simple method for computing the free energy binding strengths based on base doublets (B. Lewin, Genes VI, Oxford University Press, New York, N.Y. 1997). Isolated energy calculations are performed using a dynamic programming approach for efficiency in time.

The location of binding sites are identified by the alignment of the 16S and the target strand. Regardless of whether binding takes place or not, the location is determined by the base number where the 3′ end of the SD aligns. We ran the program on the E. coli K-12 genome with the SD mask for regions 30 bases upstream to 3000 bases downstream from the start codon for every forward coding sequence listed. The program output average binding strengths for each position relative to the respective start.

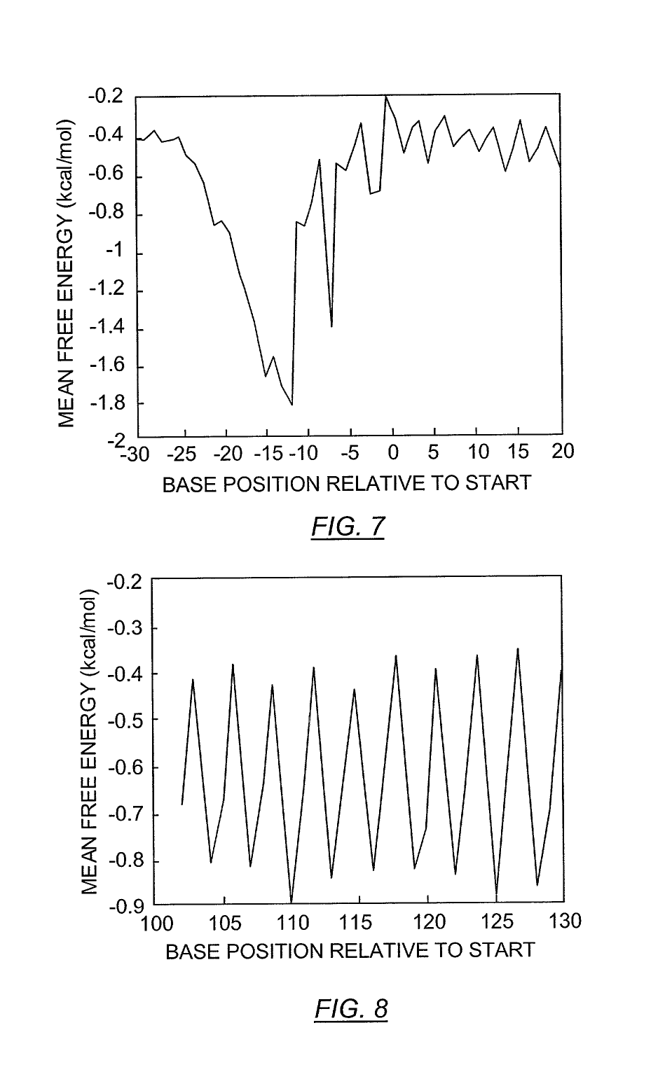

Results. The average binding strengths around the start codons are seen in FIG. 7. Average binding strengths confirm the presence of the SD sequence in the −16 to −12 positions. This region is as close to the beginning as possible without overlapping the start codon. Additionally, there is an energy spike at position −7, which corresponds to start codon affinity to the CA doublet in the SD sequence.

The average binding energies starting 102 bases downstream from the start codon are shown in FIG. 8. The binding preference is clearly seen from the sample, but we confirm with Fourier analysis of the signal.

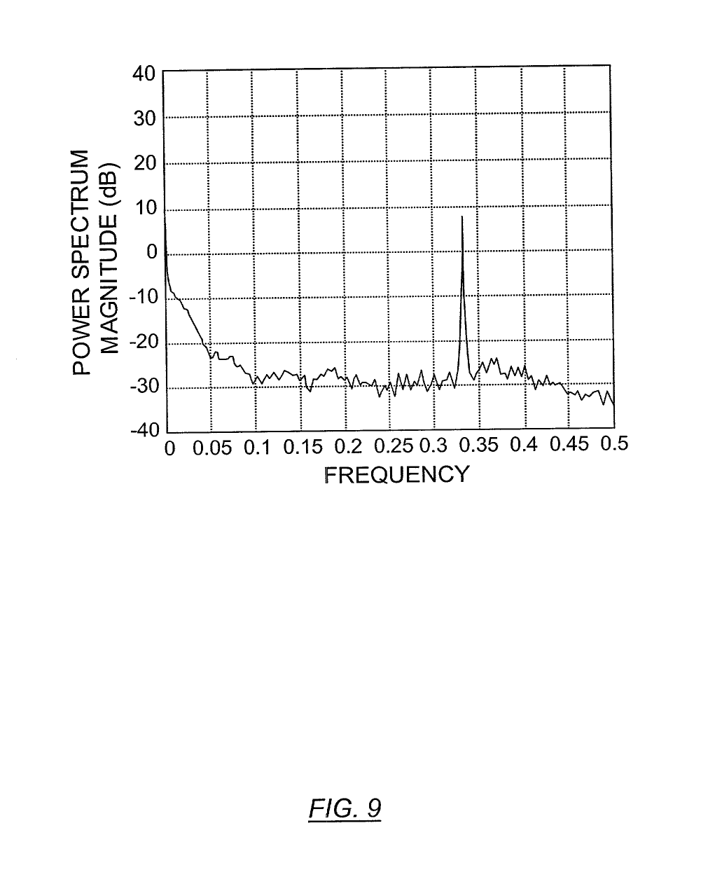

FIG. 9 shows the power spectrum density of the energy by binding position. The frequency is given in units of cycles per nucleotide. This figure shows, at frequency one-third, a peak power of 36 dB. This is equivalent to a signal to noise ratio on the order of 4,000 to 1. This indicates a pattern of binding between the SD and mRNA every third base position. Furthermore, the pattern, if it exists in individual coding sequences, must be biased toward a particular phase, otherwise the individual signals would cancel each other out. Assuming all the samples are in phase, and a 10 db per factor of ten in the number of samples, the power should be reduced in an individual sequence on the average by:

10×log₁₀2095=33 dB

Thus, an approximate signal to noise ratio of three decibels may be expected in each coding sequence on the average.

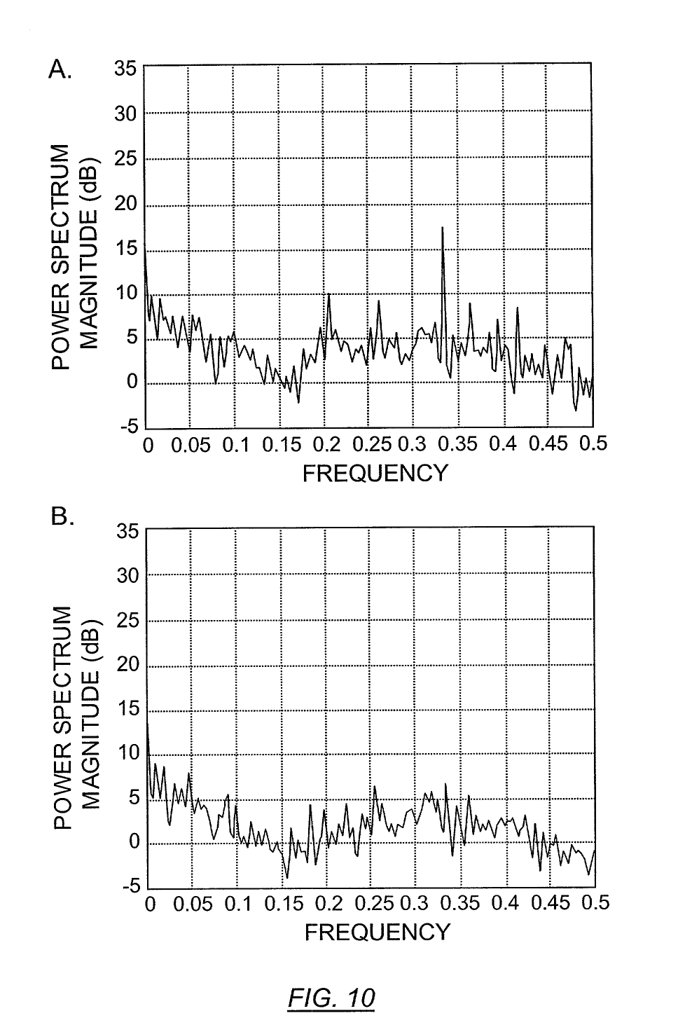

FIG. 10A, shows the spectral estimate for the gene aceF, which codes for pyruvate dehydrogenase. It has a 12 decibel signal at period three. We also compute the spectral estimate corresponding to an 1830 base long ORF unlisted in GenBank's annotation. It is located between the putative transport gene emrB, and transport gene srlA. The spectrum of the latter (shown in FIG. 10B) reveals no periodic signal of significance, let alone in the vicinity of period three.

For each coding sequence listed in GenBank, we compute the average free energy for each position modulo 3 relative to the start. The three positions (0, 1, 2) were assigned a binding pattern based on the relative averages (Strong, Medium, Weak). Table 1 shows the results of these computations. The numbers are shown for both the full CDS listing, and for the certain sequences only. Both the number of sequences, and their average length are shown.

TABLE 1
Binding Patterns: Number and Average Length.

| Pattern | Certain | Length | All | Length |
|---|---|---|---|---|
| SMW | 3 | 1042 | 24 | 440 |
| *SMW | 124 | 820 | 426 | 760 |
| **MWS | 598 | 1178 | 1570 | 1033 |
| MSW | 1 | 102 | 4 | 320 |
| WSM | 3 | 522 | 10 | 365 |
| *WMS | 13 | 426 | 61 | 549 |

Note the significant preference for stronger binding in the third positions relative to the second. 99.1% of the certain coding sequences exhibited this property, as did 97.2% of the uncertain, but suspected, coding regions.

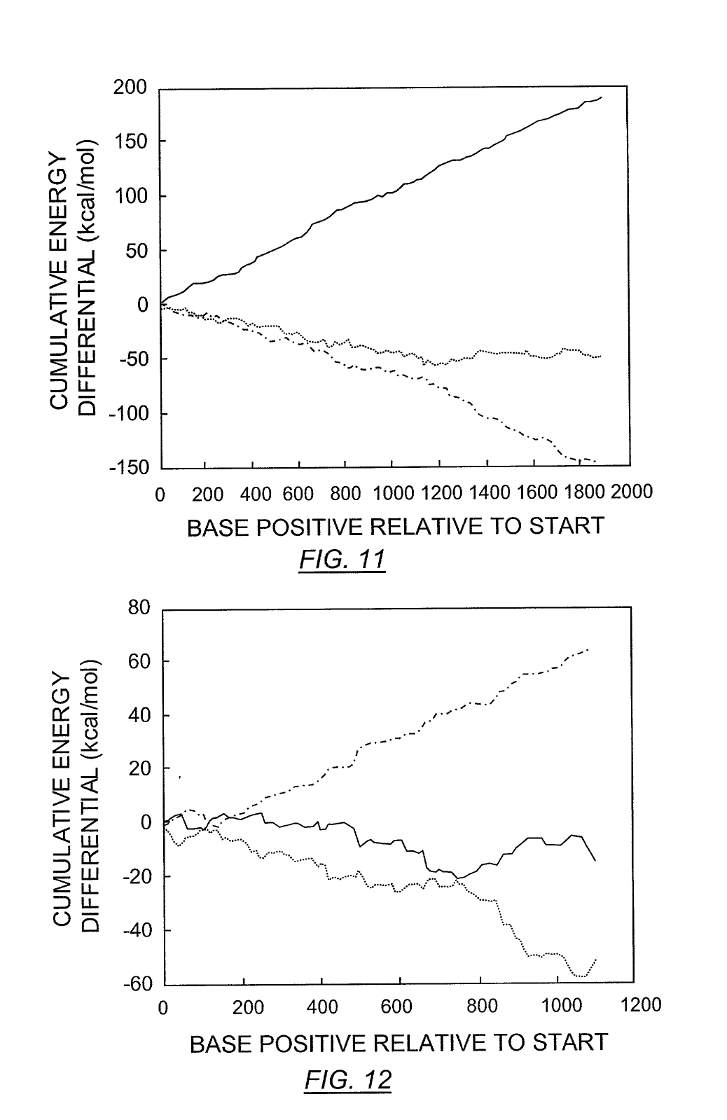

As an example of the building energy pattern, the cumulative energies in each position were calculated for aceF. The result is shown in FIG. 11. For clarity, at every point, the cumulative energy over all upstream positions is subtracted. Thus, the difference in the cumulative sum for each phase, and the overall total, is shown. The dotted line represents the accumulation of energy in the first position. The solid line represents that for the second. Note that the positive energy differential indicates weak binding. Strong binding occurs with negative free energy. The third position is seen with on the broken line. It shows the strongest binding. Thus, aceF falls into the most common category of binding patterns, MWS.

Finally, we examined the RF-2 gene prfb. The binding was computed disregarding the natural frameshift near the beginning of the sequence. We employed the same calculation for prfB as previously with aceF. The result is shown in FIG. 12. Note the change in binding preference in the vicinity of the +1 frameshift. prfb exhibits MWS binding for the first 100 or so bases, then shifts to SMW. This is identical to MWS, having skipped forward one base.

Next, we investigate the relation of to the energy signal to the actual SD sequence. What must a DNA coding sequence look like in order to produce the binding pattern of the previous section?

To answer this question, we assume that there is an ideal binding pattern, namely, MWS. If there is no bulging of the bases as they bind, each base in the DNA aligns once with each of the 13 bases in the SD. Because the base positions 0, 3, 6, 9, and 12 have neutral binding, no base preference is indicated for these positions. In positions 1, 4, 7, 10, and 13, weak binding is in order, so bases should have a tendency not to pair up in these positions. For positions 2, 5, 8, 11, and 14, the opposite is true.

For example, take the DNA base 12 downstream from the first base of the start codon. Because this base corresponds to the first base of a codon, any indication of preference made with it will correspond to the general preference of the first bases. We now convolve the SD and the DNA to extract the base distribution. For example, we start with the SD so the 5′ end (G) aligns with base 12. That leaves the 3′ end (A) with the first base of the start codon. This is binding position zero, so there is no binding preference. Advancing the SD by one base, an A now associates with base 12. Because this binding position is weak, this indicates a bias against T in the first base of a codon. Advancing once more, the U indicates a bias in favor of A.

We next apply the thirteen preference conditions for each of the three base positions to obtain an estimate of the distributions in each base position. Where no preference is indicated, we assume the bases are equally distributed. When a base is biased against, it is assumed that the distribution is equal among the remaining bases. And where there is a bias in favor, it is assumed that the base is always the desired one. For each position, the thirteen distributions are given equal weight, resulting in the final base distribution of Table 2.

TABLE 2
Base Distributions in E. coli (Percentage of Representation
by Codon Position).

| | Theoretical | | | Observed | | |
|---|---|---|---|---|---|---|
| | I | II | III | I | II | III |
| A | 30.1 | 35.9 | 12.8 | 24.7 | 30.4 | 15.9 |
| T | 14.7 | 33.3 | 25.6 | 14.0 | 28.4 | 24.0 |
| G | 35.3 | 12.8 | 35.9 | 36.1 | 18.3 | 30.6 |
| C | 19.9 | 17.9 | 25.6 | 25.2 | 22.9 | 29.4 |

The observed distribution was obtained from all the certain forward coding sequences of length 1500 nt or greater. Although the individual probabilities differ by as much as 5.5%, the relative order of the bases is conserved by position. The G/A preference over T in position 1, the A/T preference over G in position 2, and the weak representation of A in position 3 are all seen clearly in the data, despite the extreme simplicity of the model. With respect to guanine preference, this agrees with Trifonov's GHN phase bias.

Discussion. The results show that there is a strong three base period present in the coding region. They also suggest that the signal disappears in noncoding sequences. Examinations bear this out in the samples we examine. Because the DNA coding is presumed to be efficient in prokaryotic organisms, it seems unlikely that such a signal fails to serve a real purpose with regard to the actual genetics. The preference for a particular phase of the signal relative to the start codon indicates that the signal may be used to synchronize the ribosome as it travels along the coding sequence in a three base fashion as expected by the genetic code.

Furthermore, even a simple model indicates that the expected base preference parallels the observed distribution of bases, although statistics do not indicate that signal maintenance is the sole influence on the base preference.

Three major conditions must be met for proper translation of a coding sequence. First, a sufficiently long coding region is necessary for the production to be observable. Second, there must be a sufficiently strong cSD sequence upstream from the region to be translated. This may aid in initializing the energy within the ribosome as well as attract the 16S to begin construction thereof.

Third, there must be a periodic synchronization signal to maintain the reading frame. The addition of the third condition has two major consequences. First, it could be used as a major identification mechanism in DNA analysis of coding sequences. Second, it constrains the sequences that are actually codable, which then gives additional reason for the multiple codings to each amino acid.

Application of Vector Sum Energy Calculations in Determining Translational Efficiency. A vector sum energy calculation model for determining translational efficiency is described in the examples. To test the applicability of this predictive model, we referenced a paper on trangenic production. (B. E. Schoner, et. al., Proc. Natl. Acad. Sci., pages 5403-5407, 1984). In the paper, eight plasmids containing DNA coding for BGH were inserted variously into E. coli, and the percent yields were reported. Based on the supplied sequences, our program paralleled the Schoner experiments with satisfactory results.

For example, plasmids pCZ105 and pCZ110 both contain the natural BGH coding sequences, using the trp initiator. But pCZ105 contains eight additional codons between the initial ATG and the BGH code. Schoner reports a 34% yield in E. coli for the modified gene, but less that 0.5% for the unmodified.

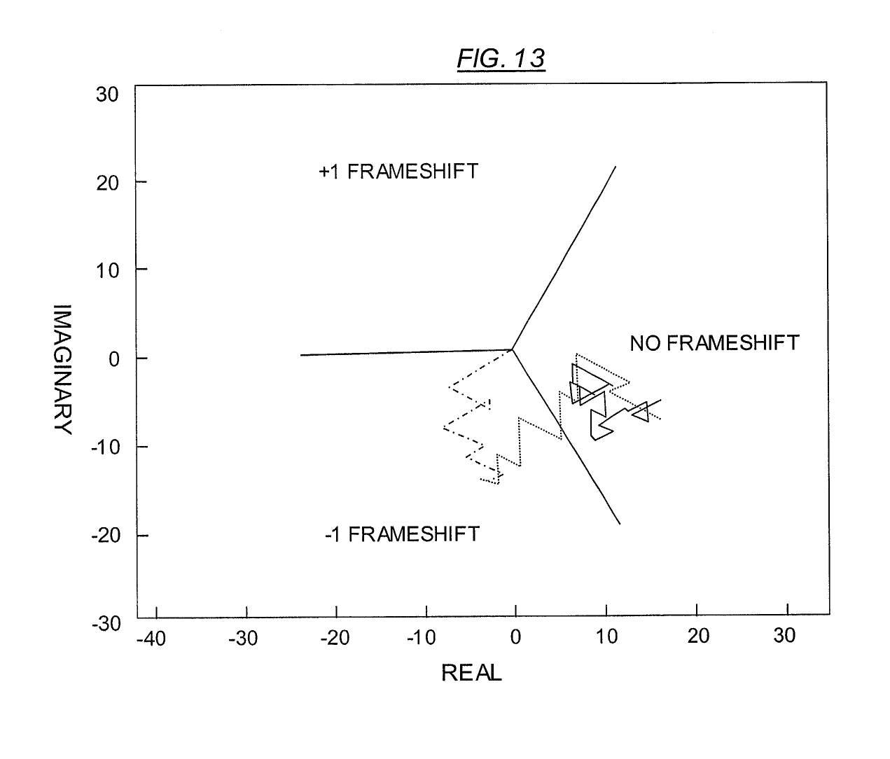

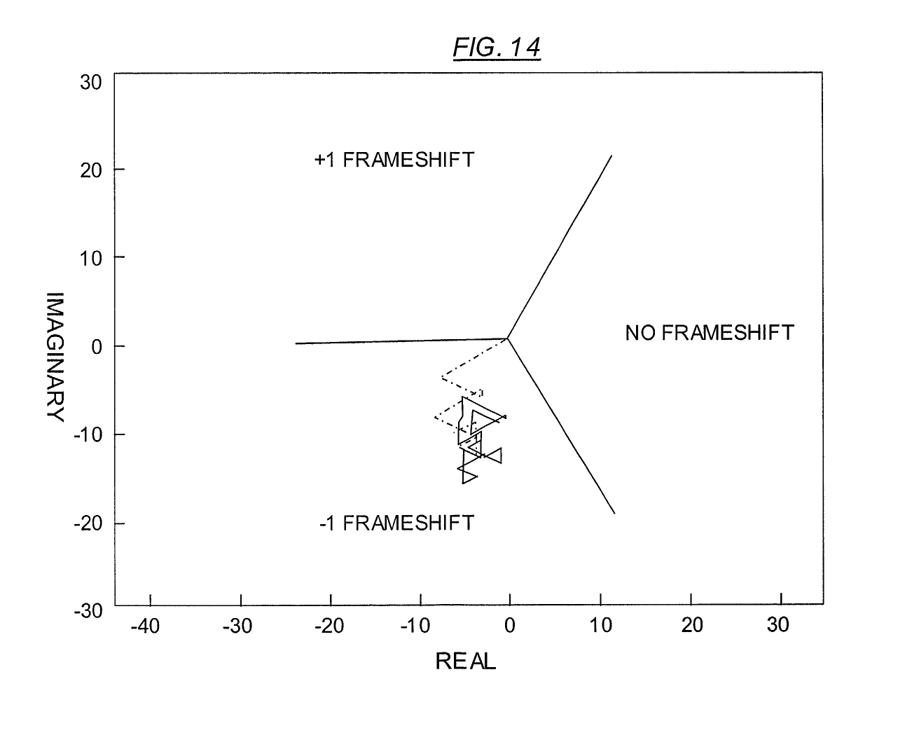

Examining the vector outputs produced by our program, (shown in FIG. 13 for pCZ105 and in FIG. 14 for pCZ110) we see that the presence of these additional codons bind in a manner that increases the stability of the translation, whereas the unmodified sequence is left in a phase prone to a −1 frameshift. This energy difference accounts for the change in translational efficiency.

### EXAMPLES

We present here an example of a method for computer-based analysis of DNA. This method provides information relating to four problems. First, to identify coding sequences within a DNA chain. Second, to predict the translational efficiency of the corresponding mRNA. Third, to optimize wobbles with respect to efficiency requirements in cross-species production, an in reverse-translation generally. Fourth, to identify the location and nature of frameshifts within the coding sequence.

While the 3′ end of the 16S rRNA is widely known to participate in initiation of translation in E. coli, the significance of continued interaction between the 3′ end and mRNA is not widely agreed upon. Preferential binding of the 16S among the three possible reading frames for a sequence may be a necessary component in message construction. We obtain desired translation data by implementing a simple computer model of ribosomal memory based on free energy calculations.

A. Methods. We now present the actual methods used.

First, knowledge of the ribosome must be obtained. In particular, the exposed sequence of bases at the 3′ end of the 16/18S rRNA must be identified.

Next, for the region of interest, we compute the binding free energy between the sequence and the exposed 3′ region at every possible location using a simple method derived from base doublet energies (See Lewin, Genes VI, Oxford University Press, New York, N.Y. 1997). We produce an energy signal based on the binding between the exposed region and an equal-length sliding window of bases in the message. The energy is computed by finding consecutive pairs of matched bases. For every such doublet pair, a negative free energy based on binding strength is obtained, and for every internal loop of mismatches, a +0.8 kcal/mol penalty is applied. The minimum of this total and zero results in the binding strength of the location. Once computed, the window shifts by a single base down the message, and the process is repeated.

B. Energy Analysis and Phase Calculation. Let the jth energy value of a binding sequence be represented by Eⱼ, starting at the 5′ end of the message. Then divide up the energy values by position modulo three, so that for r=0, 1, 2,

Eₖ^{(r)}=E₃ₖ+r (1)

Then the kth value of the rth memory register is computed iteratively as:

M_{r,k}=αM_{r,k-1}+βE₃ₖ₊ᵣ=αM_{r,k-1}+βEₖ^{(r)} (2)

Where i) 0≦α≦1 represents the degree to which memory is retained (α=0 signifying no memory, and α=1 perfect memory), ii) 0<β≦1 is an elasticity factor representing how much of the actual energy is stored at each step, and iii) for some appropriate initial condition for the memory (currently assumed to be zero at the beginning of the region of interest).

For a fixed k, the three memory registers taken together will be called collectively the memory vector,

$${\vec{M}}_{k} = [ \begin{matrix}{M}_{0 , k} \\ {M}_{1 , k} \\ {M}_{2 , k}\end{matrix} ] \qquad ( 3 )$$

The computed free energy values are necessarily nonpositive, so when α=1, the memory elements are uniformly decreasing. It is then simpler to examine the values of the memory components relative to the average. Thus, we also compute an energy differential, in which the average binding strength retained in memory is removed from the memory vector

[symbol]

ₖ=\vec{M}ₖ−\overline{M}ₖ (4)

where \overline{M}ₖ is understood in the conventional sense of this work,

$${\overline{M}}_{k} = \frac{1}{3} ( {M}_{0 , k} + {M}_{1 , k} + {M}_{2 , k} ) \qquad ( 5 )$$

C. Example Use ofMemoryModel. Suppose α=0.9, β=0.6, and {Eₖ}={−1, −2, 0, −1, −2, 0 . . . }. Then Eₖ⁽⁰⁾=−1, Eₖ⁽⁰⁾=−2, and Eₖ⁽⁰⁾=0. Initializing the memory vector, \vec{M}₀=0 allows repeated application of the register computation (equation 2).

$$\begin{matrix}{\vec{M}}_{1} = 0.9 [ \begin{matrix}0 \\ 0 \\ 0\end{matrix} ] + 0.6 [ \begin{matrix}- 1 \\ - 2 \\ 0\end{matrix} ] = [ \begin{matrix}- 0.6 \\ - 1.2 \\ 0\end{matrix} ] & ( 6 ) \\ {\vec{M}}_{2} = 0.9 [ \begin{matrix}- 0.6 \\ - 1.2 \\ 0\end{matrix} ] + 0.6 [ \begin{matrix}- 1 \\ - 2 \\ 0\end{matrix} ] = [ \begin{matrix}- 1.14 \\ - 2.28 \\ 0\end{matrix} ] & ( 7 ) \\ {\vec{M}}_{3} = 0.9 [ \begin{matrix}- 1.14 \\ - 2.28 \\ 0\end{matrix} ] + 0.6 [ \begin{matrix}- 1 \\ - 2 \\ 0\end{matrix} ] = [ \begin{matrix}- 1.626 \\ - 3.252 \\ 0\end{matrix} ] & ( 8 )\end{matrix}$$

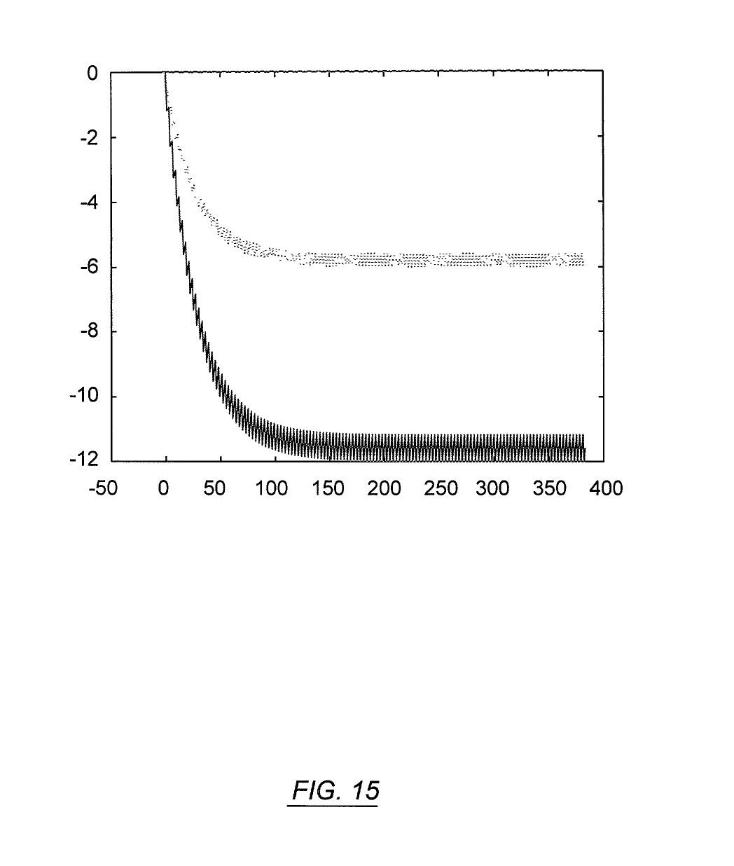

and so on. FIG. 15 shows the register contents after each energy is seen by the ribosome. The first register is in red, the second is in green, and the third in blue. When register contents or energy differentials are to be displayed in this work, the coloring convention of red, green, then blue for the three registers will be maintained. Note how the contents of the register stabilize as a consequence of the steady signal and imperfect memory. With perfect memory, the first two registers would not stabilize, but decrease without bound. The 1:2:0 ratio in input energies is maintained in memory.

The energy differential is calculated simply as

$$\begin{matrix}{{M}^{⋒}}_{1} = [ \begin{matrix}- 0.6 \\ - 1.2 \\ 0\end{matrix} ] - \frac{1}{3} ( - 0.6 - 1.2 + 0 ) = [ \begin{matrix}0 \\ - 0.6 \\ 0.6\end{matrix} ] & ( 9 ) \\ {{M}^{⋒}}_{2} = [ \begin{matrix}- 1.14 \\ - 2.28 \\ 0\end{matrix} ] - \frac{1}{3} ( - 1.14 - 2.28 + 0 ) = [ \begin{matrix}0 \\ - 1.14 \\ 1.14\end{matrix} ] & ( 10 ) \\ {{M}^{⋒}}_{3} = [ \begin{matrix}- 1.626 \\ - 3.252 \\ 0\end{matrix} ] - \frac{1}{3} ( - 1.626 - 3.252 + 0 ) = [ \begin{matrix}0 \\ - 1.626 \\ 1.626\end{matrix} ] & ( 11 )\end{matrix}$$

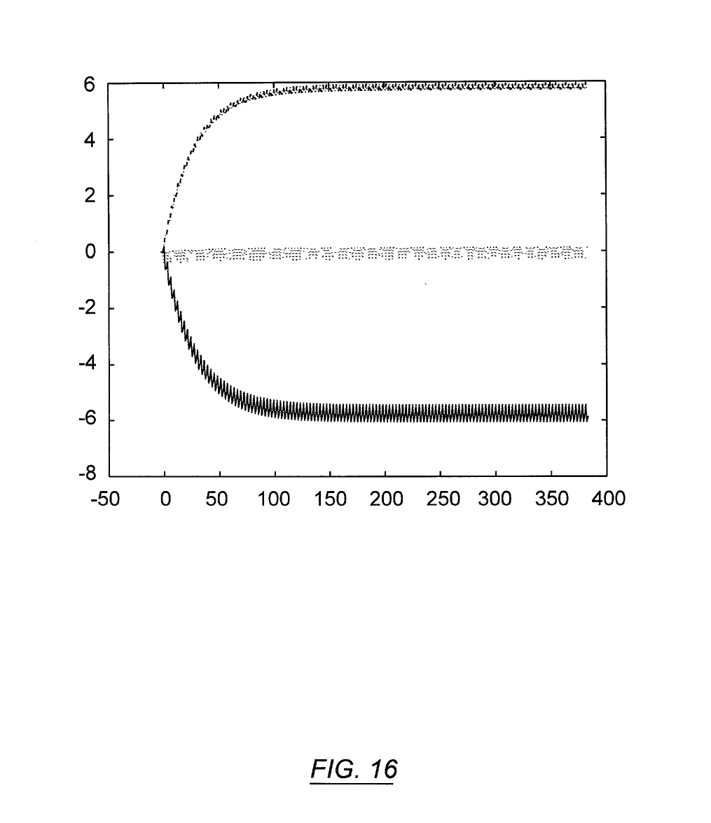

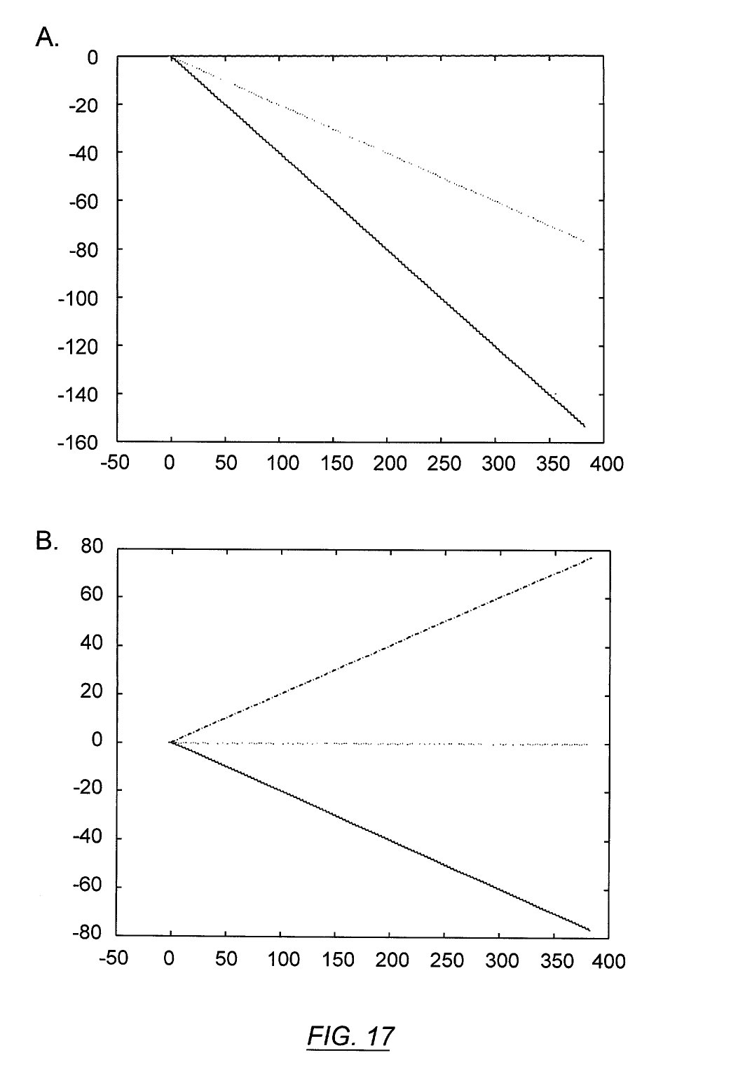

FIG. 16 shows the energy differential, color coded the same as in the previous figure. The energy differential provides less information than the actual register contents. When perfect memory is used, the differential is generally clearer. As a demonstration, FIG. 17 shows the register contents (left) and differential (right) for the same energy sequence, but with perfect memory.

D. Selection of Parameters. Qualitatively, the ration α/β represents the response rate of the memory to the binding energies. If the ratio is large, then relative energies in the ribosome will be slow to change. If the ratio is small, then the contents will be sensitive to the binding energies. However, the response rate to the signal seen by the memory mechanism is entirely determined by α.

Assume that the ribosome sees a steady signal, i.e., Eₖ^{(r)}=Eₖ₋₁^{(r)}. Then the steady-state memory in register r is

Mᵣ=αMᵣ+βEᵣ (12)

so that

$$\frac{{M}_{r}}{{E}_{r}} = \frac{β}{1 - α} \qquad ( 13 )$$

A reasonable estimate for the number of binding positions required in memory, N is 1/0.012, that is, N/3, or 1/0.036 per register. Thus,

$$\begin{matrix}{M}_{r} = α {M}_{r} + β {E}_{r} = \frac{N}{3} β {E}_{r} \approx \frac{β {E}_{n}}{0.036} & ( 14 ) \\ \frac{α {M}_{r}}{β {E}_{r}} = \frac{α}{β} \frac{β}{1 - α} = \frac{α}{1 - α} \approx \frac{1}{0.036} - 1 & ( 15 ) \\ α \approx 0.964 & ( 16 )\end{matrix}$$

Because this estimate for α indicates a fairly low level of energy dissipation in the memory, it is useful to begin by assuming perfect memory. With perfect memory, the elasticity factor, β, becomes simply one of scale, without qualitative value. That is,

$${M}_{r , k} = {M}_{r , k - 1} + β {E}_{k}^{( r )} = β {\sum }_{j = 0}^{k} {E}_{j}^{( r )} \qquad ( 17 )$$

Thus, β=1 for the time being.

By assigning each of the three memory values phase angles 2π/3 radians apart, we track the relative amounts of energy in each frame through the vector sum of the memory values. The absolute choice of phase, {θ, θ±2π/3} is arbitrary, affecting the computation by a constant rotation. It is simplest to choose the phase in correspondence with the period-three Fourier coefficient, as in:

$$\begin{matrix}{L}_{k} sin {φ}_{k} = \frac{\sqrt{3}}{2} ( {M}_{2} - {M}_{1} ) & ( 18 ) \\ {L}_{k} cos {φ}_{k} = {M}_{0} - \frac{1}{2} ( {M}_{2} + {M}_{1} ) & ( 19 )\end{matrix}$$

If the sum memory deviates sufficiently, a frameshift may be predicted. Generally, the three possible frames will divide the circle, and regions free of frameshifts will show memory in a sector 2π/3 radians wide. Depending on the base distance on the message between the rRNA 3′ binding and the actual translation site, the proper frame is selected by the phase angle. Frameshifts are indicated by translation from one 2π/3 sector to another.

E. Optimization. The preceding computations suggest a method for selecting wobbles in reverse-translation. When proper phase angle for a translated sequence is known, codons may be selected for the express purpose of maintaining the energy signal close to that phase.

A codon affects a window of n+2 binding sites, if the rRNA exposed region is n bases long. Therefore, optimization of a codon sequence may be performed codon by codon. By examining the energy pattern of the 2n−1 base long subsequence centered on the target codon, and selecting the codon that provides the optimal signal, the overall signal must be enhanced.

For example, suppose the optimal binding angle was Tc/3 radians using a ribosome with an exposed region 13 bases long. This corresponds to favored binding every third position starting with the first of the 25 base window. Recalling that negative free energies imply strong binding, the proper jth codon maximizes

$${\sum }_{i = j - 1}^{j + 3} {E}_{3 i} - {E}_{3 i + 1} + {E}_{3 i + 2} \qquad ( 20 )$$

There are many different ways to make use of the energy calculations to determine translational character and optimize wobbles, but these examples show some of the basic calculations that may be performed.

The foregoing is illustrative of the present invention, and is not to be construed as limiting thereof. The invention is defined by the following claims, with equivalents of the claims to be included therein.

That which is claimed is:

1. A computer-based method for screening a nucleic acid sequence for efficient translation in a predetermined host, said method comprising the steps of:
(a) providing a ribosomal nucleic acid sequence, said ribosomal nucleic acid sequence comprising a continuous segment of from 6 to 15 bases of the first 15 bases closest to the 3′ end of the 16S or 18S rRNA ribosomal subunit of said host;
(b) providing a substrate nucleic acid sequence;
(c) determining a first binding strength of said ribosomal nucleic acid sequence to said substrate nucleic acid sequence at a first alignment of interest in the substrate;
(d) determining a second binding strength of said ribosomal nucleic acid sequence to said substrate nucleic acid sequence at a second alignment of interest; wherein said second substrate alignment of interest is one base downstream from said first alignment of interest;
(e) determining a third binding strength of said ribosomal nucleic acid sequence to said substrate nucleic acid sequence at a third alignment of interest, wherein said third substrate alignment of interest is two bases downstream from said first alignment of interest;
(f) successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;
(g) generating a binding strength pattern from said first through third binding strengths determined in said successively repeated steps (c) through (e); and
(h) detecting the presence or absence of a three-base binding strength periodic cycle and phase through said substrate nucleic acid sequence from said binding strength pattern, the presence of said three base periodic binding strength cycle and correct phase through said substrate nucleic acid sequence indicating said substrate nucleic acid sequence is a candidate for efficient translation in said host.

2. A method according to claim 1, wherein said substrate nucleic acid sequence is heterologous to said host.

3. A method according to claim 2, wherein said substrate nucleic acid sequence is obtained from a different host than said ribosomal nucleic acid sequence.

4. A method according to claim 2, wherein said substrate nucleic acid sequence is synthetic.

5. A method according to claim 1, wherein said substrate nucleic acid sequence encodes a predetermined protein or peptide.

6. A method according to claim 1, wherein said detecting further comprises the step of determining the strength of said periodic signal.

7. A method according to claim 6, further comprising the step of:
(i) generating a quantitative indicator of translation efficiency from the strength of said periodic signal.

8. A method according to claim 6, further comprising the steps of:
(j) determining the sufficiency of said translation efficiency from said quantitative indicator; and then, in the absence of sufficient translation efficiency;
(k) replacing at least one base in said substrate nucleic acid sequence with a different base to produce a subsequent substrate nucleic acid sequence different from said previous substrate nucleic acid sequence, and encoding the same protein or peptide; and
(l) repeating steps (c) through (j) above with said subsequent substrate nucleic acid sequence.

9. A method according to claim 1, further comprising the steps of:
(j) determining the sufficiency of said translation efficiency from said quantitative indicator; and then, in the absence of sufficient translation efficiency;
(k) replacing at least one base in said substrate nucleic acid sequence with a different base to produce a subsequent substrate nucleic acid sequence different from said previous substrate nucleic acid sequence, and encoding the same protein or peptide; and
(l) repeating steps (c) through (j) above with said subsequent substrate nucleic acid sequence.

10. A method according to claim 8 or 9, wherein said step (l) further comprises repeating steps (c) through (k) until a nucleic acid sequence having sufficient efficiency of translation in said host is identified.

11. A method according to claim 1, further comprising the steps of:
detecting a phase shift in said three base periodic cycle; and
determining the presence of a frame shift in said substrate nucleic acid sequence from said phase shift, so that said substrate nucleic acid sequence remains a candidate for efficient translation in said host in the presence of said phase shift.

12. A method according to claim 1, wherein said host is a prokaryotic species.

13. A method according to claim 1, wherein said host is a eukaryotic species.

14. A method according to claim 1, further comprising the step of:
predicting the potential for proper translation of said substrate nucleic acid sequence in said host using the said already determined binding strengths of said ribosomal nucleic acid sequence to said substrate nucleic acid sequence.

15. A method according to claim 1, wherein said step (g) of generating a binding strength pattern comprises one or more calculating step(s), said calculating step(s) selected from a group comprising (i) calculating a summation of all said binding strengths, (ii) calculating a summation of said first, second, or third binding strengths, (iii) calculating an integral of all said binding strengths or of said first, second, or third binding strengths, (iv) calculating a partial integral of all said binding strengths or of said first, second, or third binding strengths, (v) calculating a running average of all said binding strengths or of said first, second, or third binding strengths, and (vi) calculating transforms of all said binding strengths or of said first, second, or third binding strengths.

16. A method according to claim 1, wherein said step (h) of detecting the presence or absence of a three-base binding strength periodic cycle comprises one or more calculating step(s), said calculating step(s) selected from a group comprising (i) calculating a summation of all said binding strengths, (ii) calculating a summation of said first, second, or third binding strengths, (iii) calculating an integral of all said binding strengths or of said first, second, or third binding strengths, (iv) calculating a partial integral of all said binding strengths or of said first, second, or third binding strengths, (v) calculating a running average of all said binding strengths or of said first, second, or third binding strengths, and (vi) calculating transforms of all said binding strengths or of said first, second, or third binding strengths.

17. A method according to claim 1, wherein said step (f) of succssively repeating steps (c) through (e) further comprising the step of:
calculating a cumulative energy for each said first, second, or third alignment of interest.

18. A method according to claim 1, further comprising the step of:
calculating a cumulative energy differential for a portion of said substrate nucleic acid sequence.

19. A method according to claim 1, further comprising the step of:
calculating a power spectrum magnitude of said binding strengths of said ribosomal nucleic acid sequence to said substrate nucleic acid sequence for a portion of said substrate nucleic acid sequence.

20. A method according to claim 1, further comprising the step of:
calculating a mean binding strength of said ribosomal nucleic acid sequence to said substrate nucleic acid sequence for a portion of said substrate nucleic acid sequence.

21. A system for screening a nucleic acid sequence for efficient translation in a predetermined host, comprising:
(a) means for providing a ribosomal nucleic acid sequence, said ribosomal nucleic acid sequence comprising a continuous segment of from 6 to 15 bases of the first 15 bases closest to the 3′ end of the 16S or 18S rRNA ribosomal subunit of said host;
(b) means for providing a substrate nucleic acid sequence;
(c) means for determining a first binding strength of said ribosomal nucleic acid sequence to said substrate nucleic acid sequence at a first alignment of interest in the substrate;
(d) means for determining a second binding strength of said ribosomal nucleic acid sequence to said substrate nucleic acid sequence at a second alignment of interest; wherein said second substrate alignment of interest is one base downstream from said first alignment of interest;
(e) means for determining a third binding strength of said ribosomal nucleic acid sequence to said substrate nucleic acid sequence at a third alignment of interest, wherein said third substrate alignment of interest is two bases downstream from said first alignment of interest;
(f) means for successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;
(g) means generating a binding strength pattern from said first through third binding strengths determined in said successively repeated steps (c) through (e); and
(h) means for detecting the presence or absence of a three-base binding strength periodic cycle and phase through said substrate nucleic acid sequence from said binding strength pattern, the presence of said three base periodic binding strength cycle and correct phase through said substrate nucleic acid sequence indicating said substrate nucleic acid sequence is a candidate for efficient translation in said host.

22. A computer program product for screening a nucleic acid sequence for efficient translation in a predetermined host, said computer program product comprising a computer usable storage medium having computer readable program code means embodied in the medium, the computer readable program code means comprising:
(a) means for providing a ribosomal nucleic acid sequence, said ribosomal nucleic acid sequence comprising a continuous segment of from 6 to 15 bases of the first 15 bases closest to the 3′ end of the 16S or 18S rRNA ribosomal subunit of said host;
(b) means for providing a substrate nucleic acid sequence;
(c) means for determining a first binding strength of said ribosomal nucleic acid sequence to said substrate nucleic acid sequence at a first alignment of interest in the substrate;
(d) means for determining a second binding strength of said ribosomal nucleic acid sequence to said substrate nucleic acid sequence at a second alignment of interest; wherein said second substrate alignment of interest is one base downstream from said first alignment of interest;
(e) means for determining a third binding strength of said ribosomal nucleic acid sequence to said substrate nucleic acid sequence at a third alignment of interest, wherein said third substrate alignment of interest is two bases downstream from said first alignment of interest;
(f) means for successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;
(g) means for generating a binding strength pattern from said first through third binding strengths determined in said successively repeated steps (c) through (e); and
(h) means for detecting the presence or absence of a three-base binding strength periodic cycle and phase through said substrate nucleic acid sequence from said binding strength pattern, the presence of said three base periodic binding strength cycle and correct phase through said substrate nucleic acid sequence indicating said substrate nucleic acid sequence is a candidate for efficient translation in said host.

23. A computer-based method for screening a nucleic acid sequence for the presence of at least one coding sequence therein, said method comprising the steps of:
(a) providing a substrate nucleic acid sequence from a predetermined host;
(b) providing a ribosomal nucleic acid sequence, said ribosomal nucleic acid sequence comprising a continuous segment of from 6 to 15 bases of the first 15 bases closest to the 3′ end of the 16S or 18S rRNA ribosomal subunit of said host;
(c) determining a first binding strength of said ribosomal nucleic acid sequence to said substrate nucleic acid sequence at a first alignment of interest;
(d) determining a second binding strength of said ribosomal nucleic acid sequence to said substrate nucleic acid sequence at a second alignment of interest; wherein said second alignment of interest is one base downstream from said first alignment of interest;
(e) determining a third binding strength of said ribosomal nucleic acid sequence to said substrate nucleic acid sequence at a third alignment of interest, wherein said third alignment of interest is two bases downstream from said first alignment of interest;
(f) successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;
(g) generating a binding strength pattern from said first through third binding strengths determined in said successively repeated steps (c) through (e); and
(h) detecting the presence or absence of a three-base binding strength periodic cycle and phase in at least one portion of the said substrate nucleic acid sequence from said binding strength pattern, the presence of said three base periodic binding strength cycle through said substrate nucleic acid sequence indicating said at least one portion of said nucleic acid sequence is a coding portion in said host.

24. A method according to claim 23, further comprising the step of:
(i) determining a start region and a stop region of said coding portion within said substrate nucleic acid sequence.

25. A method according to claim 23, wherein said detecting further comprises the step of determining the strength of said periodic signal.

26. A method according to claim 25, further comprising the step of:
(i) generating a quantitative indicator of translation efficiency from the strength of said periodic signal.

27. A method according to claim 23, further comprising the steps of:
detecting a phase shift in said three base periodic cycle; and
determining the presence of a frame shift in said substrate nucleic acid sequence from said phase shift, so that said substrate nucleic acid sequence remains a candidate for efficient translation in said host in the presence of said phase shift.

28. A method according to claim 23, wherein said host is a prokaryotic species.

29. A method according to claim 23, wherein said host is a eukaryotic species.

30. A method according to claim 23, further comprising the step of:
predicting the potential for proper translation of said substrate nucleic acid sequence in said host using the said already determined binding strengths of said ribosomal nucleic acid sequence to said substrate nucleic acid sequence.

31. A method according to claim 23, wherein said step (g) of generating a binding strength pattern comprises one or more calculating step(s), said calculating step(s) selected from a group comprising (i) calculating a summation of all said binding strengths, (ii) calculating a summation of said first, second, or third binding strengths, (iii) calculating an integral of all said binding strengths or of said first, second, or third binding strengths, (iv) calculating a partial integral of all said binding strengths or of said first, second, or third binding strengths, (v) calculating a running average of all said binding strengths or of said first, second, or third binding strengths, and (vi) calculating transforms of all said binding strengths or of said first, second, or third binding strengths.

32. A method according to claim 23, wherein said step (h) of detecting the presence or absence of a three-base binding strength periodic cycle comprises one or more calculating step(s), said calculating step(s) selected from a group comprising (i) calculating a summation of all said binding strengths, (ii) calculating a summation of said first, second, or third binding strengths, (iii) calculating an integral of all said binding strengths or of said first, second, or third binding strengths, (iv) calculating a partial integral of all said binding strengths or of said first, second, or third binding strengths, (v) calculating a running average of all said binding strengths or of said first, second, or third binding strengths, and (vi) calculating transforms of all said binding strengths or of said first, second, or third binding strengths.

33. A method according to claim 23, wherein said step (f) of succssively repeating steps (c) through (e) further comprising the step of:
calculating a cumulative energy for each said first, second, or third alignment of interest.

34. A method according to claim 23, further comprising the step of:
calculating a cumulative energy differential for a portion of said substrate nucleic acid sequence.

35. A method according to claim 23, further comprising the step of:
calculating a power spectrum magnitude of said binding strengths of said ribosomal nucleic acid sequence to said substrate nucleic acid sequence for a portion of said substrate nucleic acid sequence.

36. A system for screening a nucleic acid sequence for the presence of at least one coding sequence therein, comprising:
(a) means for providing a substrate nucleic acid sequence from a predetermined host;
(b) means for providing a ribosomal nucleic acid sequence, said ribosomal nucleic acid sequence comprising a continuous segment of from 6 to 15 bases of the first 15 bases closest to the 3′ end of the 16S or 18S rRNA ribosomal subunit of said host;
(c) means for determining a first binding strength of said ribosomal nucleic acid sequence to said substrate nucleic acid sequence at a first alignment of interest;
(d) means for determining a second binding strength of said ribosomal nucleic acid sequence to said substrate nucleic acid sequence at a second alignment of interest; wherein said second alignment of interest is one base downstream from said first alignment of interest;
(e) means for determining a third binding strength of said ribosomal nucleic acid sequence to said substrate nucleic acid sequence at a third alignment of interest, wherein said third alignment of interest is two bases downstream from said first alignment of interest;
(f) means for successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;
(g) means for generating a binding strength pattern from said first through third binding strengths determined in said successively repeated steps (c) through (e); and
(h) means for detecting the presence or absence of a three-base binding strength periodic cycle and phase in at least one portion of the said substrate nucleic acid sequence from said binding strength pattern, the presence of said three base periodic binding strength cycle through said substrate nucleic acid sequence indicating said at least one portion of said nucleic acid sequence is a coding portion in said host.

37. A computer program product for screening a nucleic acid sequence for the presence of at least one coding sequence therein, said computer program product comprising a computer usable storage medium having computer readable program code means embodied in the medium, the computer readable program code means comprising:
(a) means for providing a substrate nucleic acid sequence from a predetermined host;
(b) means for providing a ribosomal nucleic acid sequence, said ribosomal nucleic acid sequence comprising a continuous segment of from 6 to 15 bases of the first 15 bases closest to the 3′ end of the 16S or 18S rRNA ribosomal subunit of said host;
(c) means for determining a first binding strength of said ribosomal nucleic acid sequence to said substrate nucleic acid sequence at a first alignment of interest;
(d) means for determining a second binding strength of said ribosomal nucleic acid sequence to said substrate nucleic acid sequence at a second alignment of interest; wherein said second alignment of interest is one base downstream from said first alignment of interest;
(e) means for determining a third binding strength of said ribosomal nucleic acid sequence to said substrate nucleic acid sequence at a third alignment of interest, wherein said third alignment of interest is two bases downstream from said first alignment of interest;
(f) means for successively repeating steps (c) through (e) along said substrate nucleic acid sequence, wherein the first alignment of interest in each successive step (c) is three bases downstream of the first alignment of interest in the immediately preceding step (c), until a binding strength is determined at every alignment of said substrate nucleic acid sequence;
(g) means for generating a binding strength pattern from said first through third binding strengths determined in said successively repeated steps (c) through (e); and
(h) means for detecting the presence or absence of a three-base binding strength periodic cycle and phase in at least one portion of the said substrate nucleic acid sequence from said binding strength pattern, the presence of said three base periodic binding strength cycle through said substrate nucleic acid sequence indicating said at least one portion of said nucleic acid sequence is a coding portion in said host.
